(19) United States
(12) Patent Application Publication
(10) Pub. No.: US 2020/0188465 A1
(43) Pub. Date: Jun. 18, 2020

(54) TREATMENT OF CYTOKINE RELEASE SYNDROME BY DECREASING LEVEL OF PROINFLAMMATORY CYTOKINE

(71) Applicant: Gwo Xi Stem Cell Applied Technology Co., Ltd., Zhubei City (TW)

(72) Inventors: Yu-Chen Tsai, Zhubei City (TW); Chi-Ying Huang, Zhubei City (TW); Ming-Hsi Chuang, Zhubei City (TW); Po-Cheng Lin, Zhubei City (TW); Chia-Hsin Lee, Zhubei City (TW)

(21) Appl. No.: 16/710,004

(22) Filed: Dec. 11, 2019

Related U.S. Application Data
(60) Provisional application No. 62/778,318, filed on Dec. 12, 2018.

(51) Int. Cl. A61K 36/41; A61K 35/00; A61K 31/5415; A61K 31/19

(52) U.S. Cl. CPC A61K 36/41; A61K 35/00; A61K 2236/30; A61K 31/19; A61K 31/5415

(57) ABSTRACT

The present invention provides a new method for decreasing level of a proinflammatory cytokine in a subject, which is further able to treating cytokine release syndrome caused by CAR T-cell therapy or a disorder mediated by an overproduction of a proinflammatory cytokine. The method comprises administering to the subject in need thereof a therapeutically effective amount of a pharmaceutical composition which is comprising at least one selected from the group consisting of Phenothiazine derivatives, Graptopetalum paraguayense extract, Rhodiola rosea extract and Histone Deacetylase (HDAC) inhibitors.

### CROSS-REFERENCE TO RELATED APPLICATIONS

The present application claims the benefit of U.S. Provisional Patent Application No. 62/778,318 filed on Dec. 12, 2018, the entire contents of which are hereby incorporated by reference.

### BACKGROUND OF THE INVENTION

### Technical Field

The present invention relates to means for decreasing a level of a cytokine in a subject. In particular, the present invention concerns a novel method in the treatment of Cytokine Release Syndrome (CRS).

### Background

In the past, the therapeutic drugs for cancers were mainly small-molecule chemical drugs or macromolecular antibodies. Now, the therapeutic approach has reached the level of cell therapy. However, chimeric antigen receptor T cell immune therapy (CAR-T) has been widely studied. It has specific receptors and targets cells that recognize specificity, such as tumor cells. Currently, there are two pharmaceutical companies in the world, Novartis and Gilead, which have been approved by the FDA for CAR-T cell therapy. Clinical trials have shown that the therapy has high remission rate and prolonged overall survival.

Although CAR-T cell therapy is clinically effective, there are also some side effects, even death. The most prevalent adverse reaction is cytokine release syndrome (CRS). When CAR-T cells are injected into patients, T cells kill the cancer cells, and then cause the release of cytokines including INF-α, IFN-γ, IL-10, and IL-6, causing patients with fever, low blood pressure, respiratory failure. Therefore, how to control these immune storms caused by CAR-T cell reinfusion is a challenge for the CAR-T therapy.

### SUMMARY OF THE INVENTION

Therefore, after several times of drug screening and testing, the applicant find that multiple several compound or composition, such as Phenothiazine derivatives, Graptopetalum paraguayense extract, Rhodiola rosea extract and Histone Deacetylase (HDAC) inhibitors, can inhibit the production of proinflammatory cytokines, and thus provides a new method for treatment of immune response for patients while avoiding unnecessary immunosuppression due to the potential risk of diminishing antitumor efficacy.

As described above, the present invention provides a new method for decreasing level of a proinflammatory cytokine in a subject, which is further able to treating cytokine release syndrome caused by CAR T-cell therapy or a disorder mediated by an overproduction of a proinflammatory cytokine.

This is, the present invention can provide a method for decreasing level of a proinflammatory cytokine in a subject comprising administering to the subject in need thereof a therapeutically effective amount of a pharmaceutical composition which is comprising at least one selected from the group consisting of Phenothiazine derivatives, Graptopetalum paraguayense extract, Rhodiola rosea extract and Histone Deacetylase (HDAC) inhibitors.

According to one embodiment of the present invention, the proinflammatory cytokine are at least one selected from the group consisting of TNF-α, IFN-γ, IL-10, and IL-6.

According to one embodiment of the present invention, the Phenothiazine derivatives are Trifluoperazine or Thioridazine.

According to one embodiment of the present invention, the Histone Deacetylase (HDAC) inhibitor is Suberoylanilide hydroxamic acid.

According to one embodiment of the present invention, wherein IFN-γ content in the cells is reduced at least 18.6% at 6 hours after administering the pharmaceutical composition containing Thioridazine; IFN-γ content in the cells is reduced at least 28.2% or more at 24 hours after administering the pharmaceutical composition containing Thioridazine.

According to one embodiment of the present invention, wherein IFN-γ content in the cells is reduced at least 72.5% or more at hours after administering the pharmaceutical composition containing Graptopetalum paraguayense extract; IFN-γ content in the cells is reduced at least 77.7% or more at 24 hours after administering the pharmaceutical composition containing Graptopetalum paraguayense extract.

According to one embodiment of the present invention, wherein IFN-γ content in the cells is reduced at least 36.3% or more at hours after administering the pharmaceutical composition containing Rhodiola rosea extract; IFN-γ content in the cells is reduced at least 62.9% or more at 24 hours after administering the pharmaceutical composition containing Rhodiola rosea extract.

According to one embodiment of the present invention, wherein IL-6 content in the cells is reduced at least 20.7% or more at hours after administering the pharmaceutical composition containing Thioridazine; IL-6 content in the cells is reduced at least 39.5% or more at 24 hours after administering the pharmaceutical composition containing Thioridazine.

According to one embodiment of the present invention, wherein IL-6 content in the cells is reduced at least 37.5% or more at hours after administering the pharmaceutical composition containing Graptopetalum paraguayense extract; IL-6 content in the cells is reduced at least 19.4% or more at 24 hours after administering the pharmaceutical composition containing Graptopetalum paraguayense extract.

According to one embodiment of the present invention, wherein IL-6 content in the cells is reduced at least 35.2% or more at hours after administering the pharmaceutical composition containing Rhodiola rosea extract; IL-6 content in the cells is reduced at least 24.3% or more at 24 hours after administering the pharmaceutical composition containing Rhodiola rosea extract.

Another aspect of this present invention relates a method for treating cytokine release syndrome comprising administering to a subject in need thereof a therapeutically effective amount of a pharmaceutical composition which is comprising at least one selected from the group consisting of Phenothiazine derivatives, Graptopetalum paraguayense extract, Rhodiola rosea extract and Histone Deacetylase (HDAC) inhibitors.

According to one embodiment of the present invention, the cytokine release syndrome is caused by CAR T-cell therapy, and the pharmaceutical composition is administered during the CAR T-cell therapy or after the CAR T-cell therapy.

According to one embodiment of the present invention, the cytokine release syndrome involves overproduction of one or more proinflammatory cytokines.

Still another aspect of this invention relates to a method of treating a disorder mediated by an overproduction of a cytokine, such as inflammation, autoimmune diseases, diabetes, atherosclerosis and cancer.

One or more examples of the disclosure will be described in detail below in the Detailed Description of the Invention. The foregoing features of the disclosure will become more apparent from the following detailed description and the appended claims. It should be noted that the foregoing general description and the following detailed description are intended to be exemplary only for illustrative purposes and are not intended to limit the scope of the disclosure.

### BRIEF DESCRIPTION OF THE DRAWINGS

FIG. 1A is a diagram illustrating results of cell viability tests for Trifluoperazine (TFP) in the embodiment 1 of the present invention.

FIG. 1B is a diagram illustrating results of cytokine release tests for Trifluoperazine (TFP) in the embodiment 1 of the present invention.

FIG. 2A is a diagram illustrating results of cell viability tests for Thioridazine (THZ) in the embodiment 1 of the present invention.

FIG. 2B is a diagram illustrating results of cytokine release tests for Thioridazine (THZ) in the embodiment 1 of the present invention.

FIG. 3A is a diagram illustrating results of cell viability tests for Graptopetalum paraguayense extract (HH-F3) in the embodiment 1 of the present invention.

FIG. 3B is a diagram illustrating results of cytokine release tests for Graptopetalum paraguayense extract (HH-F3) in the embodiment 1 of the present invention.

FIG. 4A is a diagram illustrating results of cell viability tests for Rhodiola rosea extract (Rr-EtOH) in the embodiment 1 of the present invention.

FIG. 4B is a diagram illustrating results of cytokine release tests for Rhodiola rosea extract (Rr-EtOH) in the embodiment 1 of the present invention.

FIG. 5A is a diagram illustrating results of cell viability tests for Suberoylanilide hydroxamic acid (SAHA) in the embodiment 1 of the present invention.

FIG. 5B is a diagram illustrating results of cytokine release tests for Suberoylanilide hydroxamic acid (SAHA) in the embodiment 1 of the present invention.

FIG. 6A is a diagram illustrating results of cell viability tests for Trifluoperazine (TFP) in the embodiment 2 of the present invention.

FIG. 6B is a diagram illustrating results of cytokine release tests for Trifluoperazine (TFP) in the embodiment 2 of the present invention.

FIG. 7A is a diagram illustrating results of cell viability tests for Thioridazine (THZ) in the embodiment 2 of the present invention.

FIG. 7B is a diagram illustrating results of cytokine release tests for Thioridazine (THZ) in the embodiment 2 of the present invention.

FIG. 8A is a diagram illustrating results of cell viability tests for Graptopetalum paraguayense extract (HH-F3) in the embodiment 2 of the present invention.

FIG. 8B is a diagram illustrating results of cytokine release tests for Graptopetalum paraguayense extract (HH-F3) in the embodiment 2 of the present invention.

FIG. 9A is a diagram illustrating results of cell viability tests for Rhodiola rosea extract (Rr-EtOH) in the embodiment 2 of the present invention.

FIG. 9B is a diagram illustrating results of cytokine release tests for Rhodiola rosea extract (Rr-EtOH) in the embodiment 2 of the present invention.

FIG. 10A is a diagram illustrating results of cell viability tests for Thioridazine (THZ) in the embodiment 3 of the present invention.

FIG. 10B is a diagram illustrating results of cytokine release tests for Thioridazine (THZ) in the embodiment 3 of the present invention.

FIG. 11A is a diagram illustrating results of cell viability tests for Graptopetalum paraguayense extract (HH-F3) in the embodiment 4 of the present invention.

FIG. 11B is a diagram illustrating results of cytokine release tests for Graptopetalum paraguayense extract (HH-F3) in the embodiment 4 of the present invention.

### DETAILED DESCRIPTION OF THE PREFERRED EMBODIMENTS

Herein, all technical and scientific terms used herein have the same meaning as commonly understood by one of ordinary skill in the art to which this invention belongs. Further, unless otherwise clearly contradicted by context, singular terms used herein shall include pluralities and plural terms shall include the singular.

Unless otherwise defined herein, the term “treat, treating or treatment” means an action of administration to a patient with a particular disease or disorder, where the action can reduce the disease or disorder of the patient, or the severity of one or more symptoms, or slow or delay the progress of the disease or disorder.

Herein, the term “an effective amount” means the specific amount that can achieve the effect of decreasing level of a proinflammatory cytokine after appropriate dosing period for medical drugs directly or indirectly administrated (administered, or administration,) to patients.

Herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

Notwithstanding that the numerical ranges and parameters setting forth the broad scope of the invention are approximations, the numerical values set forth in the specific examples are presented herein as precisely as possible. Any numerical value, however, inherently contains certain errors necessarily resulting from the standard deviation found in its respective testing measurement. Herein, the term “about” generally means that an actual value is within 10%, 5%, 1%, or 0.5% above and below a particular value or range. Alternatively, the term “about” indicates that the actual value falls within an acceptable standard error of the mean when considered by one of ordinary skill in the art. Except in the Examples, or where otherwise explicitly indicated, all ranges, amounts, values, and percentages used herein (for example, for describing amounts of materials, time, temperature, operation conditions, amount ratio, and the like) are understood to be modified by the word “about”. Thus, unless expressly stated to the contrary, the numerical parameters disclosed in this specification and the appended claims are all approximations and, if required, may vary. At the very least, each numerical parameter should at least be construed in light of the number of reported significant digits and by applying ordinary rounding techniques.

In one implementation aspect of this invention, a method for decreasing level of a proinflammatory cytokine in a subject is provided by administering to the subject in need thereof a therapeutically effective amount of a pharmaceutical composition which is comprising at least one selected from the group consisting of Phenothiazine derivatives, Graptopetalum Paraguayense extract, Rhodiola rosea extract and Histone Deacetylase (HDAC) inhibitors.

In accordance with the above, the pharmaceutical composition disclosed may be prepared by well-known pharmaceutical processes. In one implementation aspect of this invention, the pharmaceutical composition disclosed in this invention may be administered by way of any appropriate dosing route, for example, systemic administration modes by oral administration as capsules, suspensions, or dragees, or by parenteral administration, for example, as intramuscular injection, intravenous injection, subcutaneous injection, or intraperitoneal injection. In addition, in some embodiments, the pharmaceutical composition disclosed in this invention may also be administered through transmucosal or transdermal means, for example, topical dermal application, or bronchial, nasal, or oral inhalation, or instillation as nasal drops; and may also be administered rectally.

For oral administration, the pharmaceutical composition disclosed in this invention may be administered with excipients or may be administered without excipients. Also, the pharmaceutical composition of this invention may also be formulated into dragees as a solid dosage form containing various auxiliaries, disintegrants, granular binders, or lubricants therein. Additionally, in an example, lactose or high molecular weight polyethylene glycols may also be used. In addition, optionally, the rate of release of any pharmaceutically active ingredient may be further improved with a coating or cladding, for example, enteric coating. In other examples, the pharmaceutical composition of this invention may also be formulated into a liposome structure or biomimetic extracellular matrix system structure, or may be filled into hard and soft gelatin capsules, or may be encapsulated into biodegradable granules in kits.

Also, in the present invention, a pharmaceutically acceptable excipient means one that is compatible with other ingredients of the pharmaceutical formulation and compatible with organisms, for example, encapsulating materials, or various additives such as absorption enhancer, antioxidant, binder, buffer, coating agent, coloring agent, diluent, disintegrant, emulsifier, supplement, filler, flavoring agent, humectant, lubricant, perfume, preservative, propellant, release agent, sterilization agent, sweeting agent, solubilizing agent, wetting agent, and mixtures thereof.

Examples of auxiliaries suitable for use in the present invention may be, for example, microcrystalline cellulose, calcium carbonate, dicalcium phosphate, or glycine. Examples of disintegrants suitable for use in the present invention may be, for example, starch, alginic ac ids, or certain silicates. Examples of granular binders suitable for use in the present invention may be, for example, polyvinylpyrrolidone, sucrose, gelatin, or acacia. Examples of granular binders suitable for use in the present invention may be, for example, magnesium stearate, sodium lauryl sulfate, or talc. Examples of excipients suitable for use in the present invention may be, for example, lactose, sucrose, mannitol, sorbitol, maize starch, wheat starch, rice starch, potato starch, gelatin, or gum tragacanth.

In some embodiments, the pharmaceutical composition of this invention is formulated into a liquid dosage form suitable for oral administration, for example, oral suspensions, emulsions, microemulsions, and/or elixirs. For such a liquid dosage form, the active ingredients of the pharmaceutical composition of this invention may be further formulated with various sweeting agents or flavoring agents, coloring agents or dyes, if desired, with addition of emulsifiers and/or suspending agents, or diluents such as water, alcohol, propylene glycol, or glycerine, or buffers used to maintain the pH.

Also, in other embodiments, the liquid formulation containing the pharmaceutical composition of this invention is made into sterile injectable solutions or suspensions; for example, made into solutions suitable for administration by intravenous, intramuscular, subcutaneous, or intraperitoneal injection.

In some embodiments, the pharmaceutical composition disclosed in this invention may be used as an additional adjunctive therapeutic agent, so as to improve the therapeutic effect of a primary treatment method of cancers such as surgery, radiotherapy, or chemotherapy. The pharmaceutical composition disclosed in this invention may be applied alone or in combination with conventional pharmaceutically acceptable auxiliaries, and, for example, may be administered orally or with a food to an individual.

In some embodiments, the method of this invention further includes before, during or after administering the pharmaceutical composition of this invention to the individual, additionally applying another primary treatment means of cancers such as surgery, radiotherapy, or chemotherapy to the individual, so as to improve the therapeutic effect of cancers in the individual.

For a more thorough and complete description of this disclosure, illustrative description for implementation aspects and specific examples of this invention is provided below; however, this is not intended to represent the only form of specific examples in which the present invention may be practiced or utilized. Features of a number of specific examples and process steps and sequences to construct and operate these specific examples are covered in the embodiments. However, the same or equivalent functions and step sequences may also be accomplished by other examples.

Firstly, standard operation processes of the tests in examples of this invention are described.

### <Cell Culture and Reagent>

Human Jurkat T cell line (Clone E6-1, ATCC TIB 152) and THP-1 were used in the embodiments and purchased from Bioresource Collection and Research Centre (BCRC), Taiwan. The cells were maintained in RPMI 1640 medium (Gibco, Carlsbad, Calif., USA) with 2 mM L-glutamine adjusted to contain 1.5 g/L sodium bicarbonate, 4.5 g/L glucose, 10 mM HEPES, 1.0 mM sodium pyruvate, 1% (v/v) penicillin-streptomycin (HyClone, Logan, Utah), and 10% (v/v) fetal bovine serum (FBS; HyClone). The cells were kept in the incubator with 5% CO₂ at 37° C. The passages were performed when the cell density didn't allow the cell concentration to exceed 3×10⁶ cells/mL.

### <Reagents and Drugs>

The ionomycin, phorbol 12-myristate 13-acetate (PMA), Lipopolysaccharide (LPS) Trifluoperazine (TFP), Thioridazine (THZ), and Suberoylanilide hydroxamic acid (SAHA) used in this embodiment were purchased from Sigma-Aldrich (St. Louis, Mo., USA).

3-(4,5-dimethylthiazol-2-yl)-5(3-carboxymethoxyphenyl)-2-(4-sulfophenyl)-2H-tetrazolium (MTS) for cytotoxicity assay was purchased from CellTilter 96® Aqueous One Solution Cell Proliferation Assay (Promega Corporation, Madison, Wis.), according to the manufacturer's instructions.

The extract of Graptopetalum paraguayense and extract of Rhodiola rosea may be prepared by the methods shown in U.S. Pat. No. 8,686,030, the disclosure of which is incorporated herein by reference in its entirety.

That is, the leaves of Graptopetalum paraguayense (referred to as GP) were ground and lyophilized into powder at −20° C. and stored in a moisture buster at 25° C. before extraction. First, 1.5 g GP powder was vortexed with 10 ml 100% methanol (MeOH) for 5 minutes and then centrifuged at 1500 g for 5 minutes. After removal of the supernatant, 10 ml H₂O, 100% acetone, 100% methanol, 100% ethanol, 70% ethanol, 50% ethanol, 100% DMSO and 30% DMSO was added to each pellet to resuspend them for each extract. The suspension was mixed by vortexing for 5 minutes, centrifuged twice at 1500 g for 5 minutes, centrifuged again at 9300 g for 5 minutes, and filtered using a 0.45 μm filter by laminar flow at room temperature.

Then, the 30% DMSO supernatant was either fractionated into four fractions (HH-F1, HH-F2, HH-F3, HH-F4) by a Sephadex LH-20 column and each fraction was further analyzed by high-performance liquid chromatography (HPLC) with a UV detector. It is preferred to choose HH-F3 in the preset invention.

Similarly, the plants of Rhodiola rosea (referred to as RS) were lyophilized into powder and stored in moisture buster at 25° C. before extraction. 1.5 grams of RS powder was dissolved in 10 ml H₂O and then centrifuged at 1500 g for 5 minutes, followed by filtering using a 0.45 lam filter by laminar flow at room temperature. The samples were stored at −20° C. as 150 mg/ml stock solutions and named as Rr-EtOH in the present invention.

### <Modeling Activation of T Cells and Cytokine Measurement>

Jurkat cells were seeded in 48-well culture plates at a density of 5.0×10⁵ cells/mL in culture medium. To promote Interferon gamma (IFN-γ; IFNG) production, the cells were stimulated with or without the combination of PMA and Ionomycin in culture medium. THP-1 cells were treated with or without the LPS that promoted IL6 production.

The culture supernatants were harvested by centrifugation and stored at −20° C. for cytokine analysis. The concentration of IFN-γ and IL6 were measured by ELISA kit (Invitrogen, Carlsbad, Calif., USA) according to the manufacturer's instructions.

### <Cell Viability Assay>

The cell viability was determined using MTS reagent (Promega). Assays were performed by adding MTS to each well for 3 hours at 37° C. with 5% CO₂. The amount of formazan converted by living cells, and then recording absorbance at 490 nm with a 96-well plate reader.

Hereafter, the present invention will be described in detail by explaining preferred embodiments of the invention with reference to the attached drawings.

### Embodiment 1

To induce IFN-γ production, the Jurkat T cells were cultured as described above and stimulated with PMA plus ionomycin for 24 hours prior to incubation with candidate drugs showed as in the Table 1 respectively. After 6 and 24 hours, the supernatants were collected for cell viability analysis and cytokine analysis. The concentration of cytokine was measured by Human IFN-γ ELISA kit (Invitrogen) according to the manufacturers.

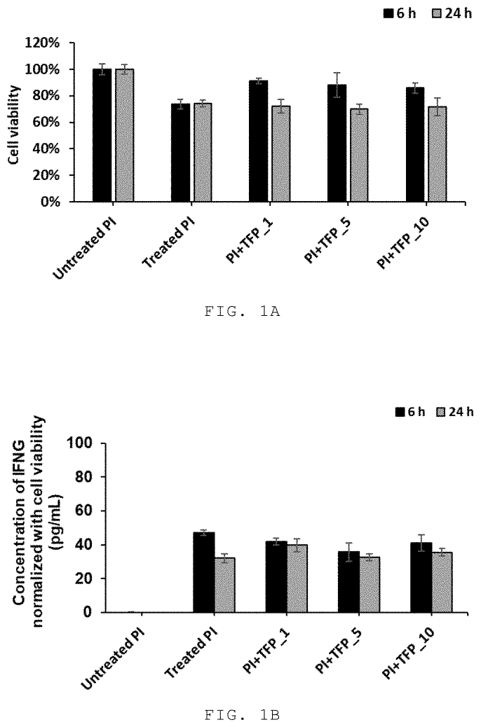

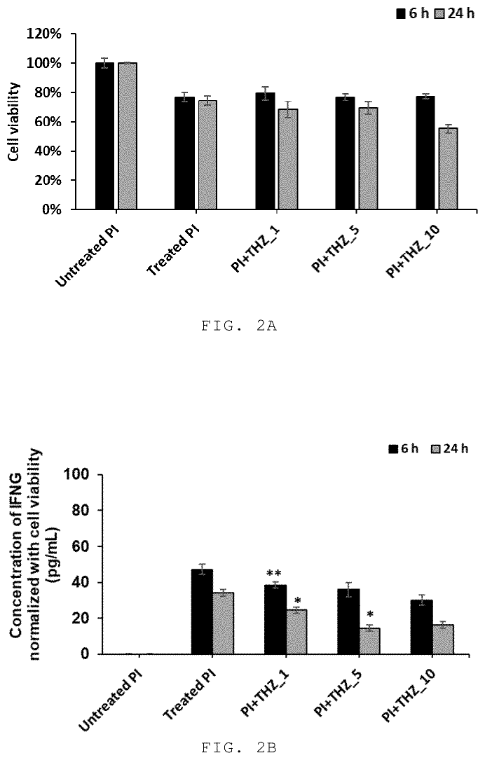

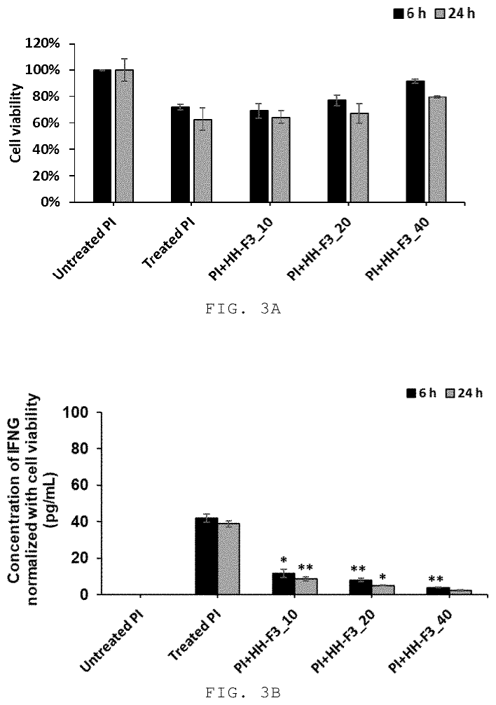

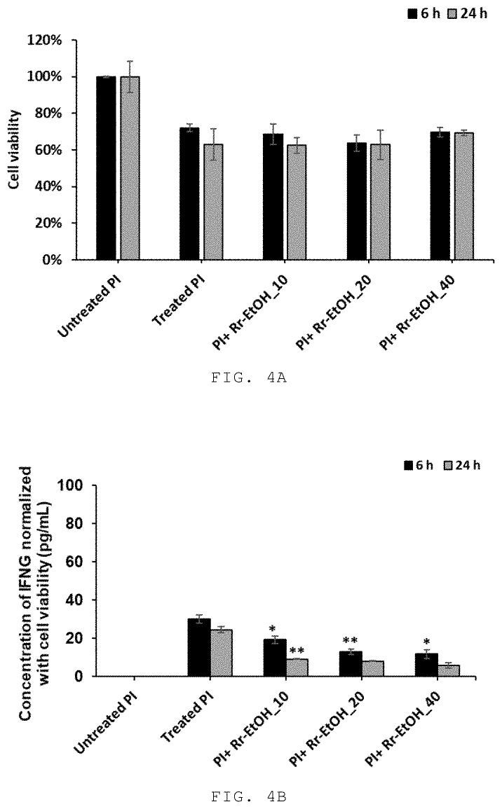

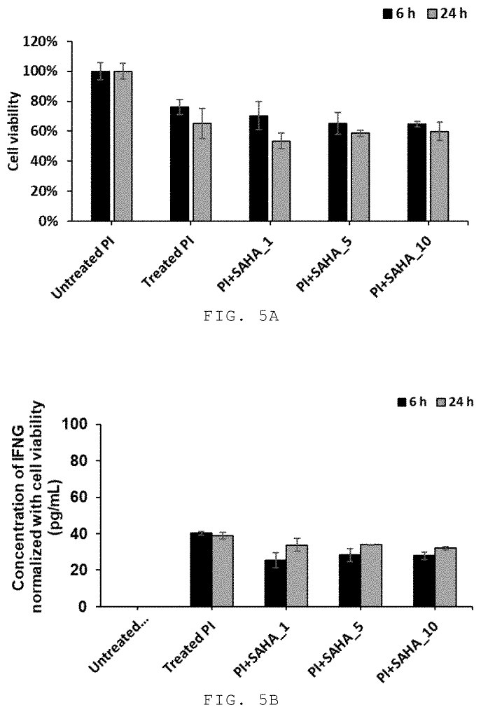

The result of the cell viability and the Concentration of IFN-γ normalized with cell viability is listed in Table 1. And the results are also showed in FIG. 1A, FIG. 1B, FIG. 2A, FIG. 2B, FIG. 3A, FIG. 3B, FIG. 4A, FIG. 4B, FIG. 5A, and FIG. 5B.

TABLE 1

| | | | | | | IFN-γ/cell | | |
|---|---|---|---|---|---|---|---|---|
| | | concentration | Cell Viability (%) | | | viability (pg/mL) | | |
| drug | group | of drugs | 0 h | 6 h | 24 h | 0 h | 6 h | 24 h |
| TFP | Untreated | 0 | 100 ± 2 | 100 ± 4 | 100 ± 3 | 0.0 | 0.0 | 0.0 |
| | PI | | | | | | | |
| | Pretreated | 0 | 85 ± 4 | 74 ± 4 | 74 ± 3 | 55.6 ± | 46.9 ± | 32.1 ± |
| | PI | | | | | 1.4 | 1.6 | 2.5 |
| | PI + TFP_1 | 1 μM | — | 91 ± 2 | 72 ± 5 | — | 41.8 ± | 39.7 ± |
| | | | | | | | 1.9 | 3.7 |
| | PI + TFP_5 | 5 μM | — | 88 ± 9 | 70 ± 4 | — | 35.7 ± | 32.5 ± |
| | | | | | | | 5.6 | 2.0 |
| | PI + TFP_10 | 10 μM | — | 86 ± 4 | 72 ± 7 | — | 40.9 ± | 35.6 ± |
| | | | | | | | 4.8 | 2.1 |
| THZ | Untreated | 0 | 100 ± 5 | 100 ± 3 | 100 ± 1 | 0.0 | 0.0 | 0.0 |
| | PI | | | | | | | |
| | Pretreated | 0 | 83 ± 5 | 77 ± 3 | 74 ± 7 | 57.3 ± | 47.2 ± | 34.0 ± |
| | PI | | | | | 2.0 | 2.8 | 1.9 |
| | PI + THZ_1 | 1 μM | — | 79 ± 5 | 69 ± 6 | — | 38.4 ± | 24.4 ± |
| | | | | | | | 1.7 | 1.9 |
| | PI + THZ_5 | 5 μM | — | 77 ± 2 | 69 ± 4 | — | 36.0 ± | 14.4 ± |
| | | | | | | | 4.0 | 1.7 |
| | PI + THZ_10 | 10 μM | — | 77 ± 2 | 55 ± 3 | — | 30.1 ± | 16.3 ± |
| | | | | | | | 2.8 | 1.8 |
| HH-F3 | Untreated | 0 | 100 ± 6 | 100 ± | 100 ± 9 | 0.0 | 0.0 | 0.0 |
| | PI | | | 0.3 | | | | |
| | Pretreated | 0 | 86 ± 7 | 72 ± 2 | 63 ± 9 | 53.8 ± | 41.9 ± | 38.8 ± |
| | PI | | | | | 4.1 | 2.2 | 1.6 |
| | PI + | 10 μg/mL | — | 69 ± 5 | 64 ± 5 | — | 11.5 ± | 8.6 ± |
| | HH-F3_10 | | | | | | 2.1 | 1.2 |
| | PI + | 20 μg/mL | — | 77 ± 4 | 67 ± 7 | — | 8.1 ± | 5.0 ± |
| | HH-F3_20 | | | | | | 1.0 | 0.2 |
| | PI + | 40 μg/mL | — | 92 ± 1 | 80 ± 1 | — | 3.9 ± | 2.2 ± |
| | HH-F3_40 | | | | | | 0.3 | 0.4 |
| Rr- | Untreated | 0 | 100 ± 4 | 100 ± | 100 ± 9 | 0.0 | 0.0 | 0.0 |
| EtOH | PI | | | 0.3 | | | | |
| | Pretreated | 0 | 85 ± 1 | 72 ± 2 | 63 ± 9 | 46.9 ± | 30.2 ± | 24.4 ± |
| | PI | | | | | 4.3 | 2.2 | 1.6 |
| | PI + | 10 μg/mL | — | 69 ± 6 | 62 ± 4 | — | 19.2 ± | 9.1 ± |
| | Rr-EtOH_10 | | | | | | 2.0 | 0.2 |
| | PI + | 20 μg/mL | — | 64 ± 4 | 63 ± 8 | — | 12.9 ± | 8.1 ± |
| | Rr-EtOH_20 | | | | | | 1.3 | 0.2 |
| | PI + | 40 μg/mL | — | 70 ± 3 | 69 ± 2 | — | 11.8 ± | 5.7 ± |
| | Rr-EtOH_40 | | | | | | 2.3 | 1.4 |
| SAHA | Untreated | 0 | 100 ± 7 | 100 ± 6 | 100 ± 5 | 0.0 | 0.0 | 0.0 |
| | PI | | | | | | | |
| | Pretreated | 0 | 81 ± 9 | 76 ± 5 | 65 ± 10 | 47.2 ± | 40.4 ± | 38.8 ± |
| | PI | | | | | 2.8 | 0.9 | 1.9 |
| | PI + SAHA_1 | 1 μM | — | 70 ± 9 | 53 ± 5 | — | 25.5 ± | 33.8 ± |
| | | | | | | | 4.1 | 3.7 |
| | PI + SAHA_5 | 5 μM | — | 65 ± 7 | 59 ± 2 | — | 28.2 ± | 34.0 ± |
| | | | | | | | 3.5 | 0.1 |
| | PI + SAHA_10 | 10 μM | — | 65 ± 2 | 60 ± 6 | — | 28.0 ± | 32.0 ± |
| | | | | | | | 2.0 | 1.0 |

IFN-γ/cell viability: Concentration of IFN-γ normalized with cell viability
Untreated PI: Medium without PMA and Ionomycin
Pretreated PI: Treatment of PMA (0.01 μg/mL) plus Ionomycin (1 μM) alone.
P: PMA; I: Ionomycin
Abbreviation:
Trifluoperazine (TFP); Thioridazine (THZ); Graptopetalum Paraguayense extract (HH-F3); Rhodiola rosea extract (Rr-EtOH); Suberoylanilide hydroxamic acid (SAHA)

Regarding the cell viability, it can be seen from Table 1 above that after 24 hours of pre-culture with PMA plus ionomycin, the cell viability of Jurkat T cells was decreased to approximately 80˜90%. And after the drugs treatment for 6 hr and 24 hr, decline of the cell viability was less than 30%, which indicating that the above drugs will not cause serious damage to the cells. Furthermore, HH-F3 is able to restore the cell damage caused by PMA and ionomycin, which increases the cell viability.

In addition, regarding the IFN-γ content, it can be seen from the above Table 1 that, Jurkat T cells can be stimulated to produce IFN-γ in the present of PMA plus ionomycin. And after the drugs treatment for 6 hr and 24 hr, the IFN-γ content in the Jurkat T cells was able to be decreased. Among them Thioridazine (THZ), HH-F3 and Rr-EtOH had stronger effects to cells compared to TFP and SAHA. The IFN-γ content is getting lower with the increase of the concentration of those three drugs and the culture time.

More specifically, IFN-γ content in the cells is respectively reduced 18.6%, 23.7%, and 36.2% after 6 hours of administering the pharmaceutical composition respectively containing 1 μM, 5 μM, and 10 μM of Thioridazine; IFN-γ content in the cells is respectively reduced 28.2%, 57.7%, and 52.2% after 24 hours of administering the pharmaceutical composition respectively containing 1 μM, 5 μM, and 10 μM of Thioridazine.

IFN-γ content in the cells is respectively reduced 72.5%, 80.8%, and 90.8% after 6 hours of administering the pharmaceutical composition respectively containing 10 μg/mL, 20 μg/mL, and 40 μg/mL of Graptopetalum paraguayense extract (HH-F3); IFN-γ content in the cells is respectively reduced 77.7%, 87.1%, and 94.4% after 24 hours of administering the pharmaceutical composition respectively containing 10 μg/mL, 20 μg/mL, and 40 μg/mL of Graptopetalum paraguayense extract (HH-F3).

IFN-γ content in the cells is respectively reduced 36.3%, 57.2%, and 60.9% after 6 hours of administering the pharmaceutical composition respectively containing 10 μg/mL, 20 μg/mL, and 40 μg/mL of Rhodiola rosea extract (Rr-EtOH); IFN-γ content in the cells is respectively reduced 62.9%, 67.0%, and 76.6% after 24 hours of administering the pharmaceutical composition respectively containing 10 μg/mL, 20 μg/mL, and 40 μg/mL of Rhodiola rosea extract (Rr-EtOH).

### Embodiment 2

To induce IL6 production, the THP-1 cells were cultured as described above and stimulated with LPS for 16 hours prior to incubation with candidate drugs showed as in the Table 2 respectively. After 6 and 24 hours, the supernatants were collected for cell viability analysis and cytokine analysis. The concentration of cytokine was measured by IL6 ELISA kit (Invitrogen) according to the manufacturers.

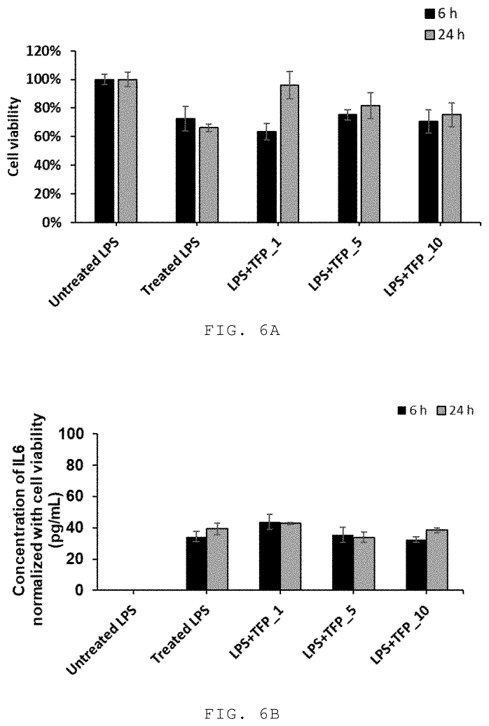

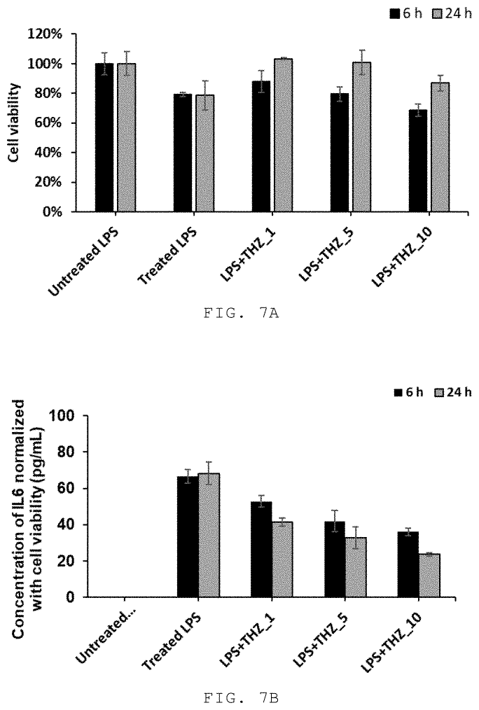

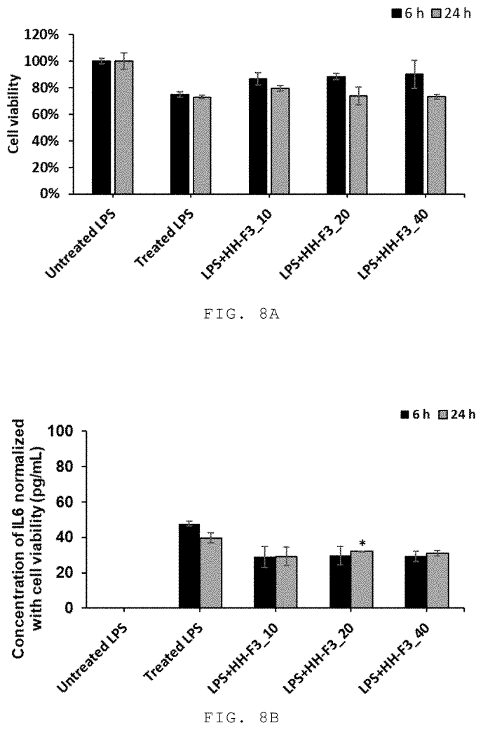

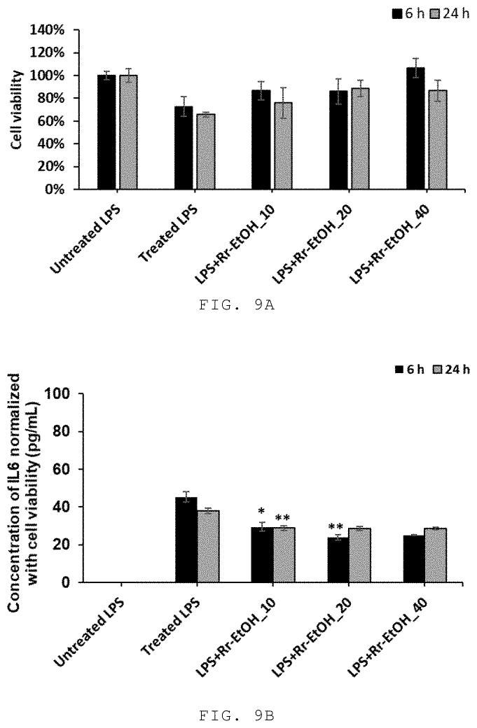

The result of the cell viability and the Concentration of IL6 normalized with cell viability is listed in Table 2. And the results are also showed in FIG. 6A, FIG. 6B, FIG. 7A, FIG. 7B, FIG. 8A, FIG. 8B, FIG. 9A, and FIG. 9B.

TABLE 2

| | | | | | | IL6/cell viability | | |
|---|---|---|---|---|---|---|---|---|
| | | concentration | Cell Viability (%) | | | (pg/mL) | | |
| drugs | group | of drugs | 0 h | 6 h | 24 h | 0 h | 6 h | 24 h |
| TFP | Untreated | 0 | 100 ± 5 | 100 ± 3 | 100 ± 5 | 0.0 | 0.0 | 0.0 |
| | LPS | | | | | | | |
| | Pretreated | 0 | 87 ± 6 | 73 ± 9 | 66 ± 3 | 42.4 ± | 34.3 ± | 39.2 ± |
| | LPS | | | | | 1.2 | 3.3 | 3.9 |
| | LPS + TFP_1 | 1 μM | | 63 ± 6 | 96 ± 9 | — | 43.9 ± | 42.8 ± |
| | | | | | | | 4.7 | 0.7 |
| | LPS + TFP_5 | 5 μM | | 75 ± 4 | 82 ± 9 | — | 35.6 ± | 33.9 ± |
| | | | | | | | 4.7 | 3.3 |
| | LPS + TFP_10 | 10 μM | | 71 ± 8 | 75 ± 8 | — | 32.5 ± | 38.4 ± |
| | | | | | | | 1.6 | 1.7 |
| THZ | Untreated | 0 | 100 ± 7 | 100 ± 7 | 100 ± 8 | 0.0 | 0.0 | 0.0 |
| | LPS | | | | | | | |
| | Pretreated | 0 | 84 ± 3 | 79 ± 1 | 78 ± 10 | 51.4 ± | 66.7 ± | 68.3 ± |
| | LPS | | | | | 8.2 | 3.9 | 6.3 |
| | LPS + THZ_1 | 1 μM | — | 88 ± 7 | 103 ± 1 | — | 52.9 ± | 41.3 ± |
| | | | | | | | 3.2 | 2.3 |
| | LPS + THZ_5 | 5 μM | — | 79 ± 5 | 101 ± 8 | — | 41.9 ± | 32.9 ± |
| | | | | | | | 5.8 | 5.9 |
| | LPS + THZ_10 | 10 μM | — | 68 ± 4 | 87 ± 5 | — | 36.1 ± | 23.7 ± |
| | | | | | | | 2.1 | 0.9 |
| HH-F3 | Untreated | 0 | 100 ± 6 | 100 ± 2 | 100 ± 6 | 0.0 | 0.0 | 0.0 |
| | LPS | | | | | | | |
| | Pretreated | 0 | 82 ± 1 | 75 ± 2 | 73 ± 1 | 42.4 ± | 47.7 ± | 39.7 ± |
| | LPS | | | | | 3.1 | 1.4 | 2.8 |
| | LPS + | 10 μg/mL | — | 87 ± 5 | 80 ± 2 | — | 29.0 ± | 29.3 ± |
| | HH-F3_10 | | | | | | 6.0 | 5.3 |
| | LPS + | 20 μg/mL | — | 88 ± 2 | 74 ± 7 | — | 29.8 ± | 32.0 ± |
| | HH-F3_20 | | | | | | 5.2 | 0.1 |
| | LPS + | 40 μg/mL | — | 90 ± 10 | 73 ± 2 | — | 29.3 ± | 30.9 ± |
| | HH-F3_40 | | | | | | 3.0 | 1.5 |
| Rr- | Untreated | 0 | 100 ± 4 | 100 ± 3 | 100 ± 6 | 0.0 | 0.0 | 0.0 |
| EtOH | LPS | | | | | | | |
| | Pretreated | 0 | 78 ± 3 | 73 ± 9 | 66 ± 2 | 53.4 ± | 45.3 ± | 38.0 ± |
| | LPS | | | | | 1.9 | 2.8 | 1.6 |
| | LPS + | 10 μg/mL | — | 87 ± 8 | 76 ± 13 | — | 29.4 ± | 28.8 ± |
| | Rr-EtOH_10 | | | | | | 2.3 | 1.1 |
| | LPS + | 20 μg/mL | — | 86 ± 11 | 89 ± 7 | — | 23.8 ± | 28.5 ± |
| | Rr-EtOH_20 | | | | | | 1.4 | 1.2 |
| | LPS + | 40 μg/mL | — | 106 ± 8 | 87 ± 9 | — | 25.1 ± | 28.7 ± |
| | Rr-EtOH_40 | | | | | | 0.2 | 0.8 |

IL6/cell viability: Concentration of IL6 normalized with cell viability
Untreated LPS: Medium without LPS
pretreated LPS: Treatment of LPS (0.1 μg/mL) alone.
Abbreviation:
Trifluoperazine (TFP); Thioridazine(THZ); Graptopetalum Paraguayense extract (HH-F3); Rhodiola rosea extract (Rr-EtOH)

Regarding the IL-6 content, it can be seen from the above Table 2 that, Jurkat T cells can be stimulated to produce IL-6 in the present of PMA plus ionomycin. And after the drugs treatment for 6 hr and 24 hr, the IL-6 content in the Jurkat T cells was able to be decreased. Among them Thioridazine (THZ), HH-F3 and Rr-EtOH had stronger effects to cells compared to TFP. The IL-6 content is getting lower with the increase of the concentration of those three drugs and the culture time.

More specifically, IL-6 content in the cells is respectively reduced 20.7%, 37.1%, and 45.9% after 6 hours of administering the pharmaceutical composition respectively containing 1 μM, 5 μM, and 10 μM of Thioridazine; IL-6 content in the cells is respectively reduced 39.5%, 51.8%, and 65.2% after 24 hours of administering the pharmaceutical composition respectively containing 1 μM, 5 μM, and 10 μM of Thioridazine.

IL-6 content in the cells is respectively reduced 39.2%, 37.5%, and 38.5% after 6 hours of administering the pharmaceutical composition respectively containing 10 μg/mL, 20 μg/mL, and 40 μg/mL of Graptopetalum paraguayense extract (HH-F3); IL-6 content in the cells is respectively reduced 26.3%, 19.4%, and 22.3% after hours of administering the pharmaceutical composition respectively containing 10 μg/mL, 20 μg/mL, and 40 μg/mL of Graptopetalum paraguayense extract (HH-F3).

IL-6 content in the cells is respectively reduced 35.2%, 47.5%, and 44.6% after 6 hours of administering the pharmaceutical composition respectively containing 10 μg/mL, 20 μg/mL, and 40 μg/mL of Rhodiola rosea extract (Rr-EtOH); IL-6 content in the cells is respectively reduced 24.3%, 24.9%, and 24.5% after 24 hours of administering the pharmaceutical composition respectively containing 10 μg/mL, 20 μg/mL, and 40 μg/mL of Rhodiola rosea extract (Rr-EtOH).

### Embodiment 3

In the Embodiment, Jurkat cells were seeded in 48-well culture plates at a density of 5.0×10⁵ cells/mL indifferent culture medium, and each of the culture medium contains PMA, ionomycin, and THZ in the proportions shown in Table 3 below.

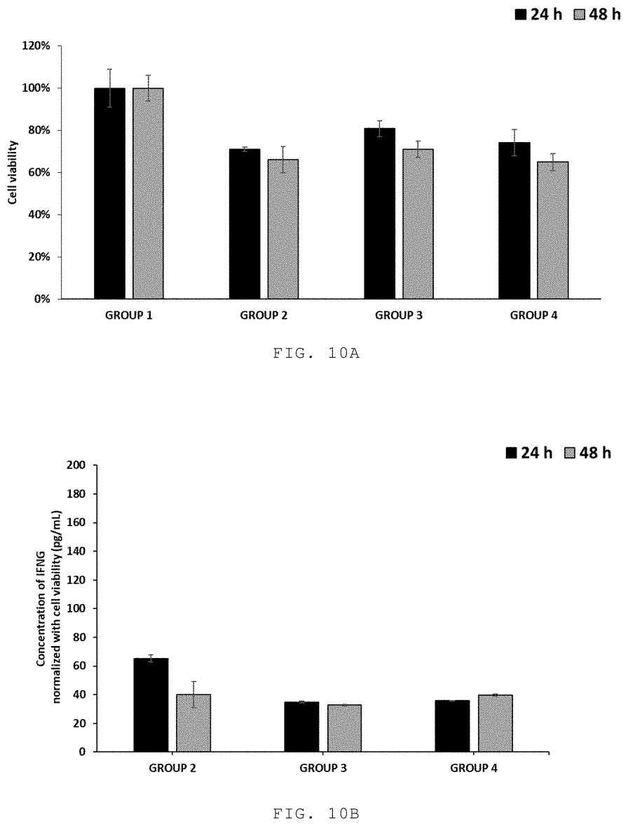

After 24 and 48 hours, the supernatants were collected for cell viability analysis and cytokine analysis. The concentration of cytokine was measured by Human IFN-γ ELISA kit (Invitrogen) according to the manufacturers. The result of the cell viability and the Concentration of IFN-γ normalized with cell viability is listed in Table 3. And the results are also showed in FIG. 10A and FIG. 10B.

TABLE 3

| | | Group | | | |
|---|---|---|---|---|---|
| | | | | 3 | 4 |
| | | 1 | 2 | (Co-treated) | (Co-treated) |
| | | (control) | (PI alone) | THZ + PI | |
| PMA (μg/mL) | | 0 | 0.01 | 0.01 | 0.01 |
| Ionomycin(μM) | | 0 | 1 | 1 | 1 |
| THZ (μM) | | 0 | 0 | 1 | 5 |
| Cell | 24 hr | 100 ± 9 | 71 ± 1 | 81 ± 4 | 74 ± 6 |
| Viability | 48 hr | 100 ± 6 | 66 ± 6 | 71 ± 4 | 65 ± 4 |
| (%) | | | | | |
| *IFN-γ/cell | 24 hr | 0.0 | 65.3 ± 2.5 | 34.9 ± 0.5 | 35.7 ± 0.2 |
| viability | 48 hr | 0.0 | 40.1 ± 9.1 | 32.6 ± 0.6 | 39.7 ± 0.6 |
| (pg/mL) | | | | | |

*IFN-γ/cell viability: Concentration of IFN-γ normalized with cell viability

From the results listed in Table 3 above, it is clearly known that the IFN-γ/cell viability of group 3 after 24 hr and 48 hr incubation were 34.9% and 32.6%, respectively, which were 46.5% and 18.7% lower than those of group 2; the IFN-γ/Cell viability of group 4 after 24 hr and 48 hr incubation were 35.7% and 39.7%, respectively, which were 45.3% and 1.0% lower than those of group 2. The co-treatment of THZ and PMA plus Ionomycin significantly attenuated cytokine induction in Jurkat cells. The results indicated that drugs could be administered to the subject during the CAR T-cell therapy prevent cytokine production.

In addition, there is no significant difference in Cell Viabilities between groups 2, 3, and 4, so it is clearly known that THZ does not affect cell viability.

### Embodiment 4

In the Embodiment, Jurkat cells were seeded in 48-well culture plates at a density of 5.0×10⁵ cells/mL in different culture medium, and each of the culture medium contains PMA, ionomycin, and HH-F3 in the proportions shown in Table 4 below.

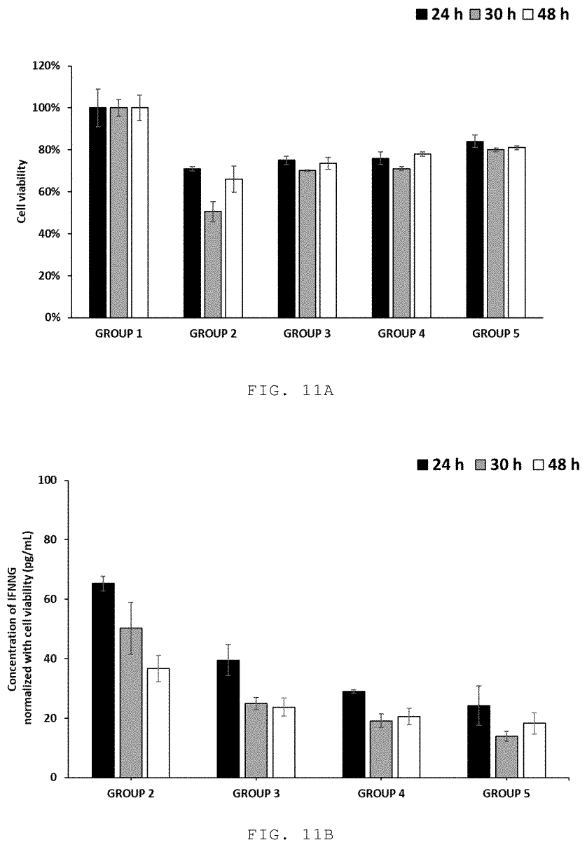

After 24, 30, and 48 hours, the supernatants were collected for cell viability analysis and cytokine analysis. The concentration of cytokine was measured by Human IFN-γ ELISA kit (Invitrogen) according to the manufacturers. The result of the cell viability and the Concentration of IFN-γ normalized with cell viability is listed in Table 4. And the results are also showed in FIG. 11A and FIG. 11B.

TABLE 4

| | | | | 3 | 4 | 5 |
|---|---|---|---|---|---|---|
| | | | | (Co- | (Co- | (Co- |
| | | 1 | 2 | treated) | treated) | treated) |
| Group | | (control) | (PI alone) | HH-F3 + PI | | |
| PMA (μg/mL) | | 0 | 0.01 | 0.01 | 0.01 | 0.01 |
| Ionomycin(μM) | | 0 | 1 | 1 | 1 | 1 |
| HH-F3(μg/mL) | | 0 | 0 | 5 | 10 | 20 |
| Cell | 24 hr | 100 ± 9 | 71 ± 1 | 75 ± 2 | 76 ± 3 | 84 ± 3 |
| Viability | 30 hr | 100 ± 4 | 51 ± 5 | 70 ± 0 | 71 ± 1 | 80 ± 1 |
| (%) | 48 hr | 100 ± 6 | 66 ± 6 | 74 ± 3 | 78 ± 1 | 81 ± 1 |
| *IFN-γ/cell | 24 hr | 0.0 | 65.3 ± 2.5 | 39.5 ± 5.3 | 29.0 ± 0.6 | 24.2 ± 0.6 |
| viability | 30 hr | 0.0 | 50.2 ± 8.7 | 24.9 ± 2.0 | 19.1 ± 2.3 | 13.9 ± 1.6 |
| (pg/mL) | 48 hr | 0.0 | 36.7 ± 4.4 | 23.7 ± 3.0 | 20.5 ± 2.7 | 18.2 ± 3.6 |

*Concentration of IFN-γ normalized with cell viability (pg/mL)

From the results listed in Table 4 above, it is clearly known that the IFN-γ/cell viability of group 3 after 24 hr, 30 hr, and 48 hr incubation were 39.5%, 24.9%, and 23.7%, respectively, which were 39.5%, 50.4%, and 35.4% lower than those of group 2; the IFN-γ/Cell viability of group 4 after 24 hr, 30 hr, and 48 hr incubation were 29.0%, 19.1%, and 20.5%, respectively, which were 55.6%, 62.0%, and 44.1% lower than those of group 2; the IFN-γ/Cell viability of group 5 after 24 hr, 30 hr, and 48 hr incubation were 24.2%, 13.9%, and 18.2%, respectively, which were 62.9%, 72.3%, 50.4% lower than those of group 2. The co-treatment of HH-F3 and PMA plus Ionomycin significantly attenuated cytokine induction in Jurkat cells. The results indicated that drugs could be administered to the subject during the CAR T-cell therapy prevent cytokine production.

Further, regarding the cell viability, the cell viabilities of groups 3 to 5 are higher than group 2, which is showing that HH-F3 can restore cell damage caused by PMA and ionomycin, and then increase the cell viability.

The present invention showed the following: The anti-psychotic drugs of Thioridazine (THZ) significantly decreased IFN-γ expression in IFN-γ-producing cells. The treatment of Chinese medicinal herbs of HH-F3 and Rr-EtOH were studied to have significant effects on reducing the amount of IFN-γ secretion, and also affected IL6 secretion in T cells. In addition, the survival rates of cells after taking treatments were almost not affected. The results suggested that candidate drugs of Thioridazine (THZ), HH-F3, and Rr-EtOH might have an immunosppressive effect, and provide potential therapy for cytokine release syndrome.

In addition, by using co-treatment of drug approach, HH-F3 and Thioridazine also are able to reduce cytokine production. The results indicated that HH-F3 and Thioridazine are prevention of cytokine release syndrome in the CAR-T cell anti-cancer therapies.

The specific embodiments described above are only used to illustrate the features and effects of the present invention, and are not intended to limit the implementation scope of present invention. Any equivalent changes and modifications made based on the content disclosed in the present invention without departing from the spirit and technical scope of the present invention still fall within the patent scope described later.

What is claimed is:

1. A method for decreasing level of a proinflammatory cytokine in a subject, comprising administering to a subject in need thereof a therapeutically effective amount of a pharmaceutical composition; wherein
the pharmaceutical composition comprises at least one selected from a group consisting of Phenothiazine derivatives, Graptopetalum paraguayense extract, Rhodiola rosea extract and Histone Deacetylase inhibitors; the Phenothiazine derivatives are Trifluoperazine or Thioridazine; and the Histone Deacetylase inhibitor is Suberoylanilide hydroxamic acid.

2. The method for decreasing level of a proinflammatory cytokine in a subject according to claim 1, wherein the proinflammatory cytokine are at least one selected from a group consisting of TNF-α, IFN-γ, IL-10, and IL-6.

3. The method for decreasing level of a proinflammatory cytokine in a subject according to claim 2, wherein the pharmaceutical composition comprises Thioridazine, Graptopetalum Paraguayense extract, and/or Rhodiola rosea extract.

4. The method for decreasing level of a proinflammatory cytokine in a subject according to claim 3, wherein IFN-γ content in cells is reduced at least 18.6% or more at 6 hours after administering the pharmaceutical composition containing Thioridazine.

5. The method for decreasing level of a proinflammatory cytokine in a subject according to claim 3, wherein IFN-γ content in cells is reduced at least 28.2% or more at 24 hours after administering the pharmaceutical composition containing Thioridazine.

6. The method for decreasing level of a proinflammatory cytokine in a subject according to claim 3, wherein IFN-γ content in cells is reduced at least 72.5% or more at 6 hours after administering the pharmaceutical composition containing Graptopetalum paraguayense extract.

7. The method for decreasing level of a proinflammatory cytokine in a subject according to claim 3, wherein IFN-γ content in cells is reduced at least 77.7% or more at 24 hours after administering the pharmaceutical composition containing Graptopetalum paraguayense extract.

8. The method for decreasing level of a proinflammatory cytokine in a subject according to claim 3, wherein IFN-γ content in cells is reduced at least 36.3% or more at 6 hours after administering the pharmaceutical composition containing Rhodiola rosea extract.

9. The method for decreasing level of a proinflammatory cytokine in a subject according to claim 3, wherein IFN-γ content in cells is reduced at least 62.9% or more at 24 hours after administering the pharmaceutical composition containing Rhodiola rosea extract.

10. The method for decreasing level of a proinflammatory cytokine in a subject according to claim 3, wherein IL-6 content in cells is reduced at least 20.7% or more at 6 hours after administering the pharmaceutical composition containing Thioridazine.

11. The method for decreasing level of a proinflammatory cytokine in a subject according to claim 3, wherein IL-6 content in cells is reduced at least 39.5% or more at 24 hours after administering the pharmaceutical composition containing Thioridazine.

12. The method for decreasing level of a proinflammatory cytokine in a subject according to claim 3, wherein IL-6 content in cells is reduced at least 37.5% or more at 6 hours after administering the pharmaceutical composition containing Graptopetalum Paraguayense extract.

13. The method for decreasing level of a proinflammatory cytokine in a subject according to claim 3, wherein IL-6 content in cells is reduced at least 19.4% or more at 24 hours after administering the pharmaceutical composition containing Graptopetalum Paraguayense extract.

14. The method for decreasing level of a proinflammatory cytokine in a subject according to claim 3, wherein IL-6 content in cells is reduced at least 35.2% or more at 6 hours after administering the pharmaceutical composition containing Rhodiola rosea extract.

15. The method for decreasing level of a proinflammatory cytokine in a subject according to claim 3, wherein IL-6 content in cells is reduced at least 24.3% or more at 24 hours after administering the pharmaceutical composition containing Rhodiola rosea extract.

16. A method for treating cytokine release syndrome comprising administering to a subject in need thereof a therapeutically effective amount of a pharmaceutical composition; wherein the cytokine release syndrome involves overproduction of one or more proinflammatory cytokines, which is caused by CAR T-cell therapy; the pharmaceutical composition is administered during the CAR T-cell therapy or after the CAR T-cell therapy and comprises at least one selected from a group consisting of Phenothiazine derivatives, Graptopetalum paraguayense extract, Rhodiola rosea extract and Histone Deacetylase inhibitors; the Phenothiazine derivatives are Trifluoperazine or Thioridazine; and the Histone Deacetylase inhibitor is Suberoylanilide hydroxamic acid.

17. The method for treating cytokine release syndrome according to claim 16, wherein proinflammatory cytokines are at least one selected from a group consisting of TNF-α, IFN-γ, IL-10, and IL-6.

18. The method for treating cytokine release syndrome according to claim 16, wherein the pharmaceutical composition comprises Thioridazine, Graptopetalum paraguayense extract, and/or Rhodiola rosea extract.
